(19) United States
(12) Patent Application Publication
(10) Pub. No.: US 2012/0290087 A1
(43) Pub. Date: Nov. 15, 2012

(54) Incus Replacement Partial Ossicular Replacement Prosthesis

(75) Inventors: Geoffrey R. Ball, Axams (AT); Michael Santek, Innsbruck (AT); Thomas Lenarz, Hannover (DE)

(73) Assignee: VIBRANT MED-EL HEARING TECHNOLOGY GMBH, Innsbruck (AT)

(21) Appl. No.: 13/556,264

(22) Filed: Jul. 24, 2012

Related U.S. Application Data
(62) Division of application No. 13/009,104, filed on Jan. 19, 2011.
(60) Provisional application No. 61/296,928, filed on Jan. 21, 2010.
(60) Provisional application No. 61/365,824, filed on Jul. 20, 2010.

(51) Int. Cl. A61F 2/18

(52) U.S. Cl. 623/10

(57) ABSTRACT

An ossicular prosthesis is described which includes an elongated prosthesis member formed from a single foldable plane structure and having a proximal end and a distal end. A cochlear striker surface is located at the distal end of the prosthesis member for engaging an outer cochlear surface of a recipient patient. A transducer clamp is at the proximal end of the prosthesis member and includes multiple clamping fingers for securely engaging the outer surface of an enclosed acoustic signal transducer such that acoustic vibration of the signal transducer is coupled by the prosthesis member to the cochlear surface

This application is a divisional of U.S. patent application Ser. No. 13/009,104, filed Jan. 19, 2011, which in turn claims priority from U.S. Provisional Patent Application 61/296,928, filed Jan. 21, 2010, and from U.S. Provisional Patent Application 61/365,824, filed Jul. 20, 2010, which are incorporated herein by reference.

### FIELD OF THE INVENTION

The present invention relates to medical implants, and more specifically to a novel ossicular prosthesis arrangement.

### BACKGROUND ART

A normal ear transmits sounds as shown in FIG. 1 through the outer ear 101 to the tympanic membrane (eardrum) 102, which moves the ossicles of the middle ear 103 (malleus, incus, and stapes) that vibrate the oval window and round window openings of the cochlea 104. The cochlea 104 is a long narrow duct wound spirally about its axis for approximately two and a half turns. It includes an upper channel known as the scala vestibuli and a lower channel known as the scala tympani, which are connected by the cochlear duct. The cochlea 104 forms an upright spiraling cone with a center called the modiolar where the spiral ganglion cells of the acoustic nerve 105 reside. In response to received sounds transmitted by the middle ear 103, the fluid-filled cochlea 104 functions as a transducer to generate electric pulses which are transmitted to the cochlear nerve 105, and ultimately to the brain.

Hearing is impaired when there are problems in the ability to transduce external sounds into meaningful action potentials along the neural substrate of the cochlea 104. To improve impaired hearing, auditory prostheses have been developed. For example, when the impairment is related to operation of the middle ear 103, a conventional hearing aid may be used to provide acoustic-mechanical stimulation to the auditory system in the form of amplified sound.

Middle ear implants also have been developed that employ electromagnetic transducers to mechanically stimulate the structures of the middle ear 103. A coil winding is held stationary by attachment to a non-vibrating structure within the middle ear 103 and a microphone signal current is delivered to the coil winding to generate an electromagnetic field. A magnet is attached to an ossicle within the middle ear 103 so that the magnetic field of the magnet interacts with the magnetic field of the coil. The magnet vibrates in response to the interaction of the magnetic fields, causing vibration of the bones of the middle ear 103. See U.S. Pat. No. 6,190,305, which is incorporated herein by reference.

Middle ear implants using electromagnetic transducers can present some problems. Many are installed using complex surgical procedures which present the usual risks associated with major surgery and which also require disarticulating (disconnecting) one or more of the bones of the middle ear 103. Disarticulation deprives the patient of any residual hearing he or she may have had prior to surgery, placing the patient in a worsened position if the implanted device is later found to be ineffective in improving the patient's hearing.

### SUMMARY OF THE INVENTION

Embodiments of the present invention are directed to an ossicular prosthesis which includes an elongated prosthesis member formed from a single foldable plane structure and having a proximal end and a distal end. A cochlear striker surface is located at the distal end of the prosthesis member for engaging an outer cochlear surface of a recipient patient. A transducer clamp is at the proximal end of the prosthesis member and includes multiple clamping fingers for securely engaging the outer surface of an enclosed acoustic signal transducer such that acoustic vibration of the signal transducer is coupled by the prosthesis member to the cochlear surface.

The cochlear surface may include the round window membrane and/or the oval window membrane of the cochlea. The prosthesis member may be made of titanium. And the prosthesis member may also include a locking clamp having: i. a clamp strap having a fixed end and a free end, and ii. a locking head at the fixed end of the clamp strap and having a strap opening for insertion of the free end of the clamp strap, wherein the clamp strap passes around an ossicle of the middle ear in a closed loop and is fixedly engaged by the locking head for fixedly attaching the prosthesis to the ossicle

### BRIEF DESCRIPTION OF THE DRAWINGS

FIG. 1 shows various anatomical structures of a normal human ear.

FIG. 2A shows one embodiment of an ossicular prosthesis in an open position.

FIG. 2B shows another embodiment of an ossicular prosthesis in a closed position.

FIG. 3A shows another embodiment of an ossicular prosthesis in a closed position.

FIG. 3B and 3C shows the ossicular prosthesis of FIG. 3A in situ in a recipient patient.

FIG. 4A shows an elevated perspective view of another embodiment of an ossicular connector for coupling an acoustic signal transducer to an ossicle in the middle ear.

FIG. 4B shows the ossicular prosthesis of FIG. 4A in situ in a recipient patient.

FIG. 5 A-C shows perspective views of various alternative embodiments of an ossicular connector.

FIG. 6A shows a single foldable plane structure for forming a middle ear prosthesis.

FIG. 6B shows the structure of FIG. 6A as folded into an open middle ear prosthesis ready to be surgically attached.

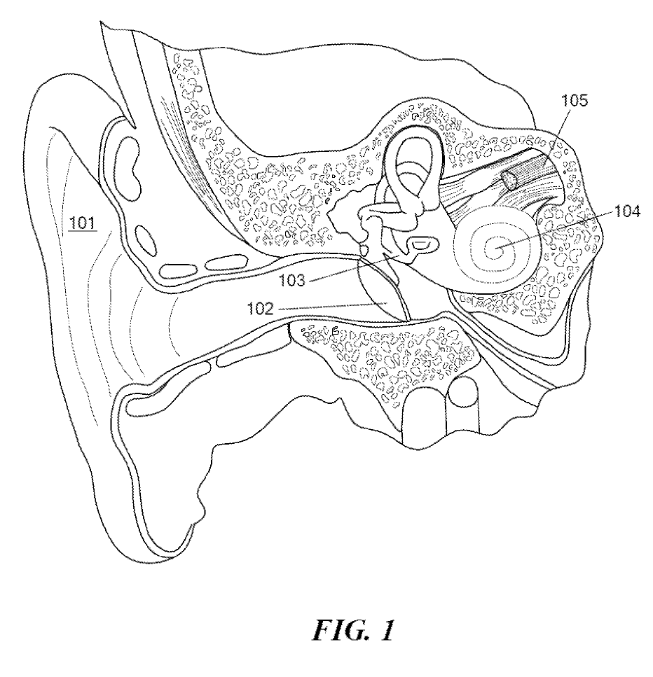

### DETAILED DESCRIPTION OF SPECIFIC EMBODIMENTS

Various embodiments of the present invention are directed to an ossicular prosthesis specifically referred to as a partial ossicular replacement prosthesis (PORP). The PORP uses a locking clamp having a clamp strap to connect a prosthesis member to an ossicle so as to couple vibration from the ossicle to the outer cochlea surface of a recipient patient for otologic reconstruction thereby providing sound sensation to the patient.

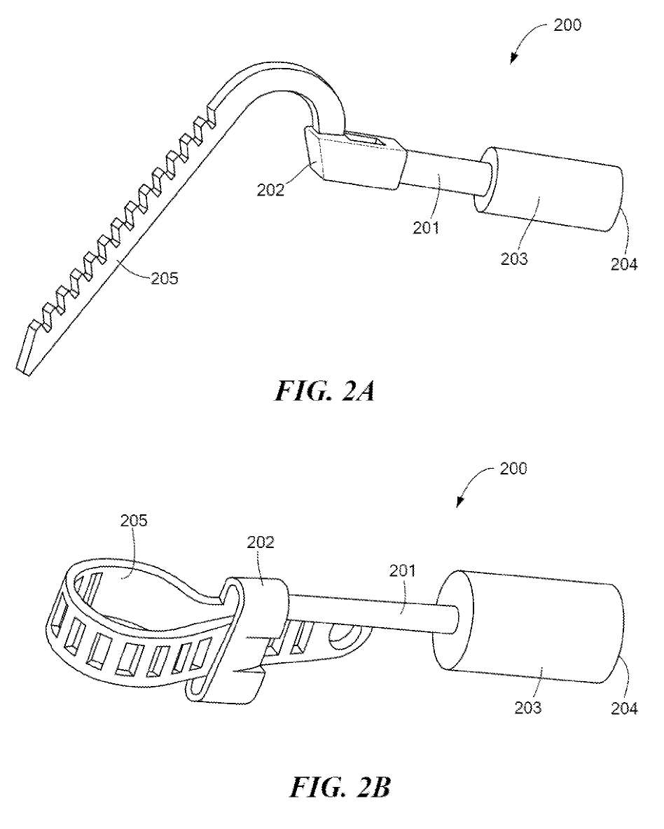

FIG. 2 A-B shows two examples of a PORP ossicular prosthesis 200 which includes an elongated titanium prosthesis member 201 having a proximal end and a distal end. A cochlea striker mass 203 is at the distal end of the prosthesis member 201 and includes an outer striking surface 204 for coupling vibration of the striker mass 203 to an outer cochlea surface of a recipient patient. A locking clamp is at the proximal end of the prosthesis member 201 and includes a clamp strap 205 having a fixed end and a free end, and a locking head 202 at the fixed end of the clamp strap 205. The locking head 202 has a strap opening for insertion of the free end of the clamp strap 205. Thus, the locking clamp is basically a cable-tie or zip-lock type clamp which because the size of loop is adjustable is a one size fits all device.

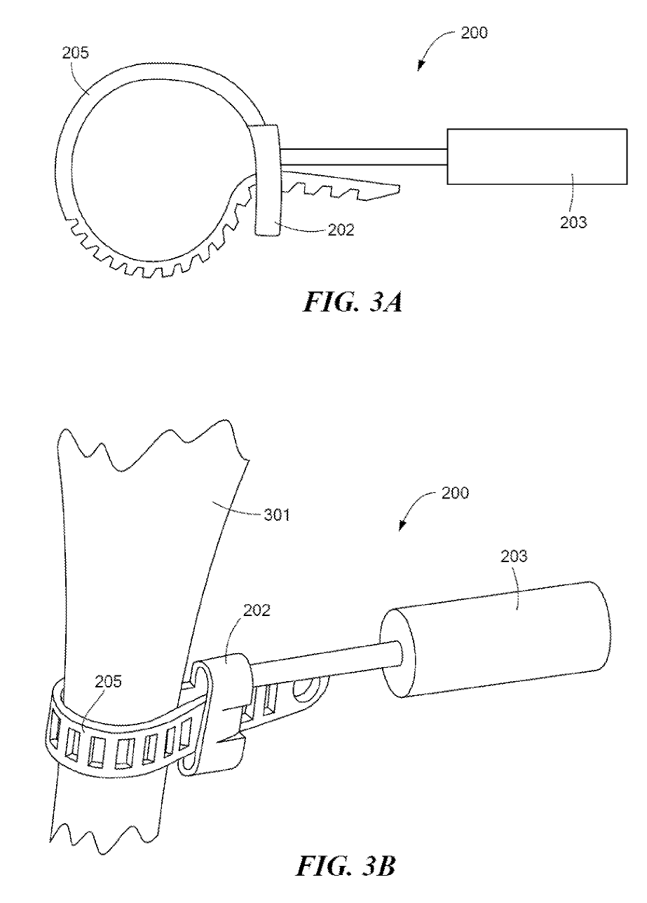

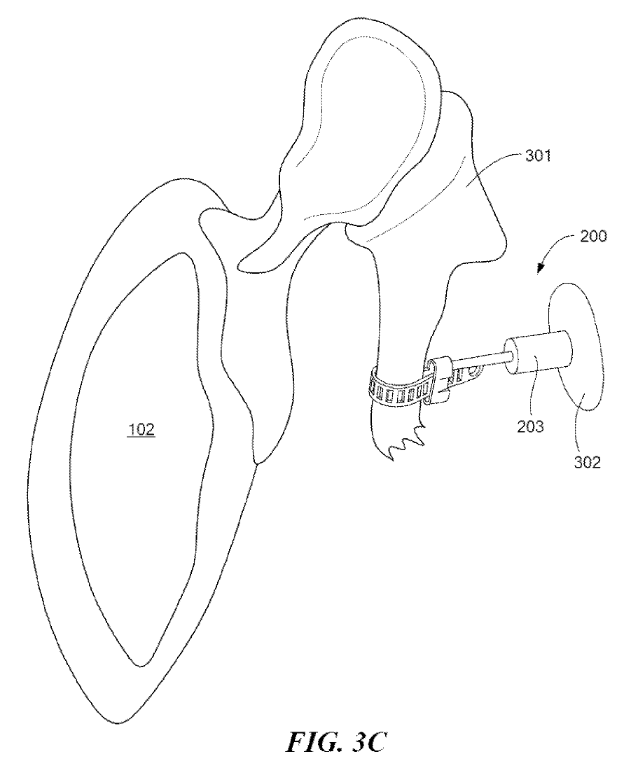

FIG. 3A shows a top view of a PROP ossicular prosthesis 200 as described above. FIG. 3B shows in close detail how the clamp strap 205 of the ossicular prosthesis 200 passes around an ossicle 301 of the middle ear (e.g., a disarticulated incus) in a closed loop and is fixedly engaged by the locking head 202 such that acoustic vibration of the ossicle 301 is coupled by the ossicular prosthesis 200 to a cochlea surface 302 of the patient such as the oval window membrane and/or the round window membrane. The clamp strap 205 is made of plastic or a polymer material so it can be tightened around the ossicle 301 and pulled snug with a desired amount of tension that can be dialed in by the surgeon. FIG. 3C shows the arrangement of FIG. 3A and 3B in situ in a recipient patient.

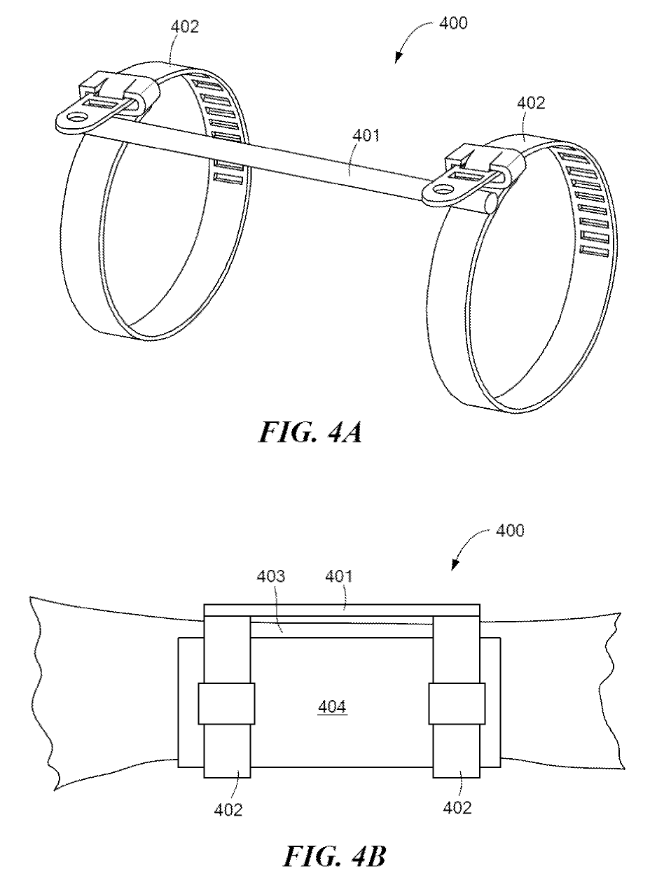

FIG. 4 A-B shows another embodiment of an ossicular connector 400 for coupling an acoustic signal transducer such as an implanted floating mass transducer (FMT) to an ossicle in the middle ear. The ossicular connector 400 includes an elongated titanium connector member 401, at least one of which as a locking clamp 402 which includes a clamp strap and a locking head. In the embodiment shown in FIG. 4 A-B, there actually is a locking clamp 402 at each end of the connector member 401. The clamp strap of the locking clamp 402 passes around an ossicle 403 of the middle ear (e.g., a disarticulated incus) and an implanted acoustic signal transducer 404 in a closed loop for attaching the signal transducer 404 to the ossicle 403 such that acoustic vibration of the signal transducer 404 is coupled to the ossicle 403, and/or vice versa. The locking clamp 402 may specifically be a cable tie-type clamp. The clamp strap may be made of plastic or a polymer material.

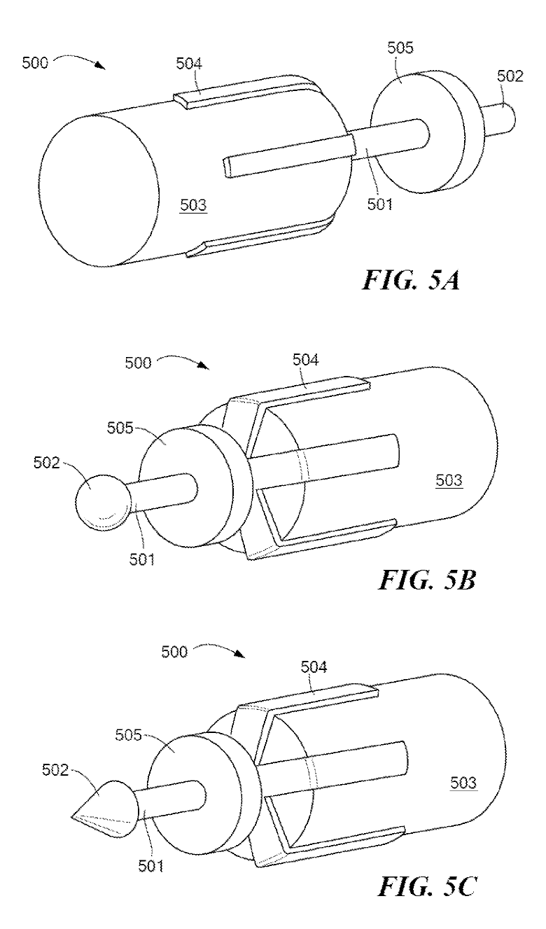

FIG. 5 A-C shows other embodiments of a middle ear prosthesis 500 for coupling an acoustic signal transducer 503 such as an FMT to the ossicular chain, for example, an ossicular bone or the outer surface of a patient cochlea such as the oval window or round window membranes. The middle ear prosthesis 500 includes an elongated titanium prosthesis member 501 with a proximal end and a distal end. A cochlea striker mass 502 is at the distal end of the prosthesis member 501 and has an outer striking surface for coupling vibration of the striker mass 502 to the ossicular chain of a recipient patient. For example, as shown in FIG. 5 A-C, the striking surface of the striker mass 502 may specifically be rod-shaped, conical, or spherical. The striker mass 502 may also include a resilient disc 505.A transducer clamp is at the proximal end of the prosthesis member 501 and includes clamping fingers 504 for securely engaging the outer surface of an enclosed acoustic signal transducer 503 such that acoustic vibration of the signal transducer 503 is coupled by the prosthesis member 501 to the ossicular chain.

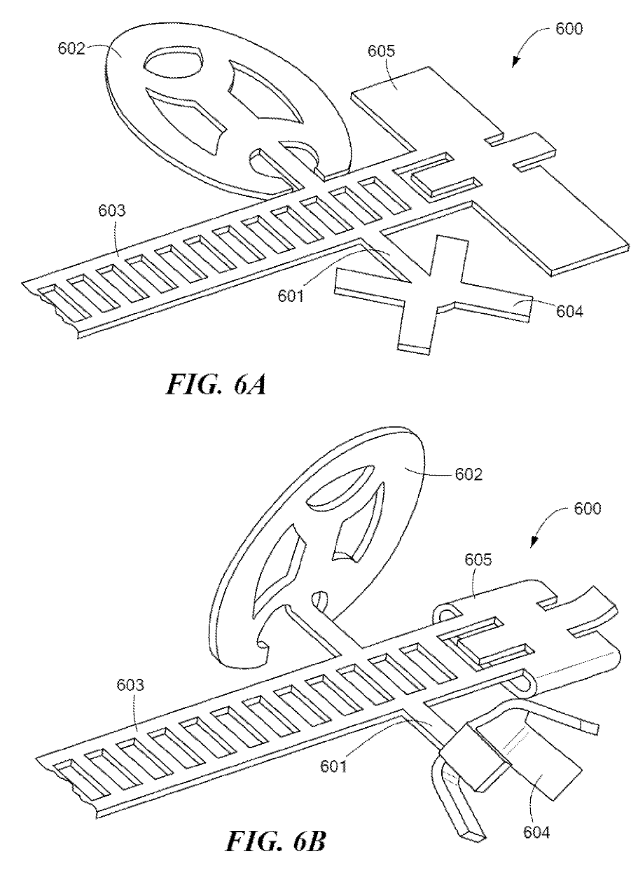

FIG. 6 A-B shows another embodiment of a middle ear prosthesis 600 for based a structure formed from a single folded plane. FIG. 6 A shows an example of a single planar sheet metal structure which is chemically etched as shown to have foldable sections 601-605. Following the chemical etching of the metal plate into the desired shape, it may be bent with one or more manufacturing fixtures into the desired shapes as shown in FIG. 6 B. In specific embodiments, some or all of a middle ear prosthesis 600 may be formed from such a single foldable plane structure: the prosthesis member 601 and the striker mass 602, the prosthesis member 601 and the transducer clamp 604, or the entire prosthesis 600 may be formed from a single foldable plane structure.

Embodiments of the present invention may be useful more generally in other surgeries for repair of structures where a clamp is called for, such as for limb repair other than in the middle ear. For example, a prosthesis member using a cable-tie type locking clamp to connect to a limb in some cases may avoid the need to use titanium screws. Similarly, a prosthetic strut may be implemented with a cable-tie type locking mechanism at either or both ends.

Although various exemplary embodiments of the invention have been disclosed, it should be apparent to those skilled in the art that various changes and modifications can be made which will achieve some of the advantages of the invention without departing from the true scope of the invention.

What is claimed is:

1. A middle ear prosthesis comprising:
an elongated prosthesis member formed from a single foldable plane structure and having a proximal end and a distal end;
a cochlear striker surface at the distal end of the prosthesis member for engaging an outer cochlear surface of a recipient patient; and
a transducer clamp at the proximal end of the prosthesis member including a plurality of clamping fingers for securely engaging the outer surface of an enclosed acoustic signal transducer such that acoustic vibration of the signal transducer is coupled by the prosthesis member to the cochlear surface.

2. A middle ear prosthesis according to claim 1, wherein the cochlear surface includes the round window membrane of the cochlea.

3. A middle ear prosthesis according to claim 1, wherein the cochlear surface includes the oval window membrane of the cochlea.

4. A middle ear prosthesis according to claim 1, wherein the prosthesis member is made of titanium.

5. A middle ear prosthesis according to claim 1, wherein the prosthesis member further comprises:
a locking clamp having:
i. a clamp strap having a fixed end and a free end, and
ii. a locking head at the fixed end of the clamp strap and having a strap opening for insertion of the free end of the clamp strap;
wherein the clamp strap passes around an ossicle of the middle ear in a closed loop and is fixedly engaged by the locking head for fixedly attaching the prosthesis to the ossicle.
